(19) United States
(12) Patent Application Publication
(10) Pub. No.: US 2017/0095316 A1
(43) Pub. Date: Apr. 6, 2017

(54) METHOD FOR CLEANING AND MAINTAINING A MEDICAL, IN PARTICULAR DENTAL, TREATMENT INSTRUMENT

(71) Applicant: KALTENBACH & VOIGT GMBH, Biberach (DE)

(72) Inventor: Hans-Dieter Wiek, Hochdorf (DE)

(21) Appl. No.: 15/278,948

(22) Filed: Sep. 28, 2016

(30) Foreign Application Priority Data
Oct. 1, 2015 (EP) 15 187 929.3

(51) Int. Cl. A61B 90/70; A61C 19/00

(52) U.S. Cl. CPC A61B 90/70; A61C 19/002

(57) ABSTRACT

In accordance with the invention, method for cleaning and maintaining a medical, in particular dental, treatment instrument is provided, in which the instrument is cleaned with the aid of a cleaning agent (I) and is subsequently maintained by means of an oil-containing maintenance agent (V). In this connection, prior to supplying the oil-containing maintenance agent (V) in the first instance the cleaning agent is substantially completely removed in a rinsing step (III). As a result of the removal of the cleaning agent, it is possible to achieve a situation where the oil-containing maintenance agent can have a better effect. In particular, it is thus possible to reduce the risk that residues of the cleaning agent, which are still located in the interior of the treatment instrument after the cleaning process, negatively impair effective lubrication at sliding and bearing points of the treatment instrument by the oil-containing maintenance agent.

The invention relates to a method for cleaning and maintaining a medical, in particular dental, treatment instrument, in which the instrument is cleaned with the aid of a cleaning agent and is subsequently maintained by means of an oil-containing maintenance agent.

A medical or dental treatment instrument is typically a substantially tubular portion which a doctor or dentist grips during the treatment. A treatment instrument usually used in dental practice is a so-called drilling handpiece which bears a treatment tool, in particular a drill, at its front end and which is coupled to a supply hose by means of a coupling at its rear end. The treatment tool can be exchanged and is held by means of a clamping system.

Typically, supply lines for energy to drive the treatment instrument and also fluid lines for treatment media, for example air and/or water, extend through the handpiece. A distinction is made in this case as a rule between so-called turbine handpieces, in which compressed air is provided to supply a turbine arranged in the front end region, and so-called motor handpieces, which have an electric motor as a drive unit.

In order to sustain the function of such a treatment instrument, from time to time conditioning or maintenance, for example of rotatably mounted elements of the instrument, is required. Furthermore, the ever increasing hygiene demands in dental practice result in treatment instruments having to be conditioned at regular intervals in time.

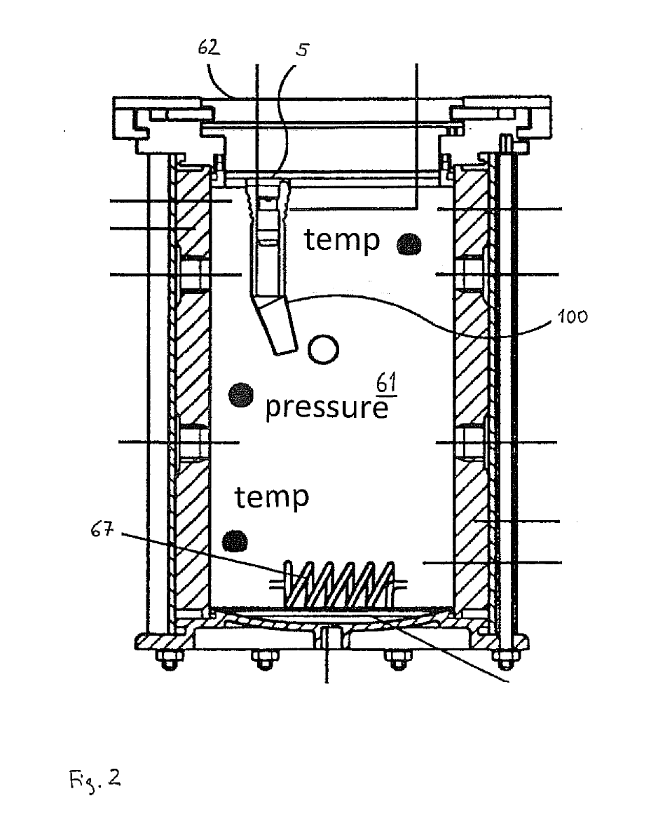

A device for conditioning dental treatment instruments is known from WO 2011/101396 A1. The device comprises a chamber or maintenance chamber 61, which is sketched in FIG. 2, in which an instrument 100 that is to be conditioned can be arranged with the aid of a coupling 5. In a first step, a cleaning agent is then flushed through an interior space of the treatment instrument 100. In this way, proteins, saliva and other organic impurities can be removed from the interior of the instrument. In a further step, the interior space of the instrument is then treated with an oil maintenance agent. As indicated in FIG. 2, the chamber 61 can be sealable with a lid 62 and comprise a heating means 67.

Practical experience has now shown that a corresponding treatment instrument after such conditioning can sometimes no longer be operated perfectly; in particular in the case of a drilling handpiece the result can be that the clamping system, with which the drill is held, can only be operated with very great difficulty.

The underlying object of the invention is to specify an improved method for cleaning and maintaining a medical, in particular dental, treatment instrument. In particular, by means of the method the risk that the treated instrument can no longer be operated perfectly is to be reduced.

This object is achieved in accordance with the invention with the subject matter mentioned in the independent claim. Particular embodiments of the invention are specified in the dependent claims.

In accordance with the invention, method for cleaning and maintaining a medical, in particular dental, treatment instrument is provided, in which the instrument is cleaned with the aid of a cleaning agent and is subsequently maintained by means of an oil-containing maintenance agent. In this connection, prior to supplying the oil-containing maintenance agent in the first instance the cleaning agent is substantially completely removed in a rinsing step.

As a result of the removal of the cleaning agent it is possible to achieve a situation where the oil-containing maintenance agent can have a better effect. In particular, it is thus possible to reduce the risk that residues of the cleaning agent, which are still located in the interior of the treatment instrument after the cleaning process or step, negatively impair effective lubrication at sliding or bearing points of the treatment instrument by the oil-containing maintenance agent.

In this connection, in the rinsing step the instrument is preferably rinsed with water. As a result, the cleaning agent can be removed in a particularly effective manner, in particular if the water is deionized water.

After the rinsing step prior to supplying the oil-containing maintenance agent, the instrument is preferably cleaned with steam. As a result of the heating of the instrument that is effected in this case, in particular it is possible to achieve a situation where the oil-containing maintenance agent can be applied in a particularly suitable manner.

After the supply of the cleaning agent prior to the rinsing step in the first instance this cleaning agent is preferably at least partly removed out of or from the instrument by means of compressed air. As a result, the cleaning agent can be removed in the following rinsing step in a particularly suitable effective manner.

The oil-containing maintenance agent preferably contains an emulsifier. As a result, in particular suitable lubrication can be achieved at components of the treatment instrument that are under water or at which residues of water have accumulated.

The invention is explained in greater detail in the following with the aid of an exemplary embodiment and with reference to the drawings, in which:

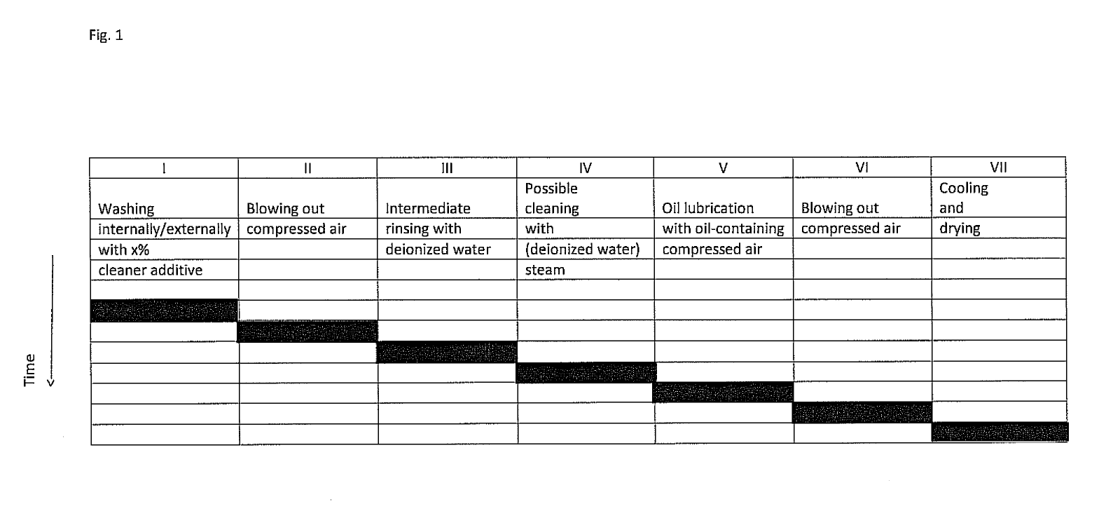

FIG. 1 shows a table of the sequence of a method in accordance with the invention; and

FIG. 2 shows a sketch of a device for carrying out a method in accordance with the invention.

The cause of the possible malfunction described at the beginning at a medical or dental treatment instrument conditioned with an automatic conditioning machine is seen in the fact that residues of the cleaning agent in the region of sliding or bearing points in the interior of the treatment instrument prevent suitable lubrication by the oil-containing maintenance agent. This is particularly so if the cleaning agent contains surfactants. Even if the treatment instrument—here also called “instrument” for short—is evacuated with compressed air blown through it after the cleaning step or is dried by means of the supply of heat, at the sliding or bearing points mentioned residues of the cleaning agent can still be found that impair a subsequent build-up of a suitable lubricating film by the oil-containing maintenance agent. This applies in particular if the points mentioned are in a non-dry state, that is, are still affected by water.

In order to carry out a method in accordance with the invention, in particular a device can be provided as known as such from WO 2011/101396 A1 mentioned at the beginning. As mentioned above, this device—as sketched in FIG. 2—can have in particular a chamber 61 in which the instrument 100 that is to be conditioned can be arranged at a coupling 5. Unless otherwise specified, the device can be configured in accordance with the representation in WO 2011/101396 A1, to the contents of which express reference is made here in this respect.

In accordance with the invention, the method for cleaning and maintaining a corresponding instrument 100 thus comprises a cleaning step, denoted in the table of FIG. 1 by “I”, in which the instrument is cleaned with the aid of a cleaning agent. In particular, the method is suitable if the instrument 100 has an interior space in which at least one sliding or bearing point is located. Such a sliding or bearing point can, for example, be part of a clamping system of the instrument 100 for holding a treatment tool, for example a drill. In the following it is assumed that a plurality of such sliding or bearing points are present in the interior of the instrument.

In the case of the cleaning step, accordingly preferably the interior space of the instrument, in particular the sliding or bearing points, is/are then cleaned with the cleaning agent. The cleaning agent is then preferably directed by way of the coupling 5 into the interior space.

In addition, in this case it is possible to provide for an outer surface of the instrument to be cleaned.

In particular, the cleaning agent can contain surfactants.

In a rinsing step III, which is carried out chronologically after the cleaning step I, the cleaning agent is at least substantially completely removed, in particular out of the interior space, in particular from the sliding or bearing points.

In this rinsing step III rinsing of the instrument, in particular of its interior space, is preferably effected with water, preferably with deionized water. In this connection it can be provided for the water to be supplied by way of the coupling 5. Particularly preferably, water is used in this case that has no additive at all. As a result, the cleaning agent can be removed in a particularly suitable manner.

For example in this connection it can be provided that in rinsing step III rinsing with the water is carried out for at least 15 seconds.

In an oil step V, which is carried out chronologically after the rinsing step III, the instrument, in particular its interior space, in particular the sliding or bearing points, is/are maintained with an oil-containing maintenance agent. In this connection, it is preferably provided that the oil-containing maintenance agent is transported into the interior space of the instrument or to the sliding or bearing points with the aid of compressed air, in particular by way of the coupling 5.

The oil-containing maintenance agent preferably contains an emulsifier.

After the rinsing step III and prior to the oil step V the instrument, in particular its interior space, is preferably cleaned in a steam-cleaning step IV with steam. This is preferably steam from deionized water; the supply of the steam is in turn preferably effected by way of the coupling 5.

Heating of the interior space or of the sliding or bearing points can be brought about by means of the steam-cleaning step IV, leading to a situation where in the following oil step the oil-containing maintenance agent is applied to the sliding or bearing points in a particularly suitable manner.

Preferably after the cleaning step I and prior to the rinsing step III the cleaning agent is partly removed out of or from the instrument, in particular out of the interior space of the instrument or the sliding or bearing point, by means of compressed air in a blow-out step II. As a result, it is possible to increase the effect of the removal of the cleaning agent that occurs in the following rinsing step III. The compressed air in this case is preferably again directed into the interior space in a corresponding manner by way of the coupling 5.

Furthermore, advantageously it can be provided that after the oil step V a further blow-out step VI is effected by supplying compressed air into the interior space of the instrument, preferably by way of the coupling 5. After this further blow-out step VI, a cooling and drying step can then additionally be carried out.

1. A method for cleaning and maintaining a medical treatment instrument, the method comprising:
cleansing the medical instrument with the aid of a cleaning agent;
subsequently maintaining the medical instrument by supplying an oil-containing maintenance agent; and
prior to supplying the oil-containing maintenance agent in a first instance, rinsing the medical instrument to substantially completely remove the cleaning agent.

2. The method according to claim 1,
wherein rinsing the medical instrument to substantially completely remove the cleansing agent includes rinsing the medical instrument with water.

3. The method according to claim 2,
further comprising after the rinsing the medical instruments and prior to supplying the oil-containing maintenance agent, cleaning the instrument with steam.

4. The method according to claim 1,
further comprising prior to rinsing the medical instrument step in the first instance, at least partly removing the cleaning agent out of or from the medical instrument by means of compressed air.

5. The method according to claim 1,
wherein the oil-containing maintenance agent contains an emulsifier.

6. The method according to claim 2,
further comprising prior to rinsing the medical instrument step in the first instance, at least partly removing the cleaning agent out of or from the medical instrument by means of compressed air.

7. The method according to claim 3,
further comprising prior to rinsing the medical instrument step in the first instance, at least partly removing the cleaning agent out of or from the medical instrument by means of compressed air.
